(19) United States
(12) Patent Application Publication
(10) Pub. No.: US 2005/0272141 A1
(43) Pub. Date: Dec. 8, 2005

(54) Anti-graffiti paint formulations and removal

(76) Inventor: Ian Crawford, Queensland (AU)

Correspondence Address: MCCARTER & ENGLISH LLP;CITYPLACE I, 185 ASYLUM STREET, HARTFORD, CT, 06103, US

(21) Appl. No.: 10/362,612

(22) PCT Filed: Aug. 17, 2001
(86) PCT No.: PCT/AU01/01022

§ 371 Date: May 25, 2005

(30) Foreign Application Priority Data
Aug. 22, 2000 (AU) PQ 9591
Aug. 31, 2000 (AU) PQ9849

(51) Int. Cl. C12N 9/20; D06M 16/00; C08L 89/00

(52) U.S. Cl. 435/264; 435/198; 524/17

(57) ABSTRACT

A paint formulation which contains specific enzymes which remain inactive in an applied surface coating of the paint formulation until specifically activated by a subsequently-applied applicator solution to facilitate removal of the surface coating. When the applicator is applied to the surface coating, the enzymes catalyse the hydrolysis of the binder in the paint or surface coating, e.g. by cleaving the ester bonds in the 1 and 3 positions of the triglyceride molecules. This allows for easy and rapid removal of the coating without damaging or affecting the underlying surface. The invention is useful either for general purpose paint removal, or for the cleaning up or removal of unwanted graffiti.

### FIELD OF THE INVENTION

The present invention relates to paint or surface coating formulations having a built-in removal factor or component. More specifically, the present invention relate to enzyme preparations for inclusion in alkyd and oil paints and a rapid removal activator applicator designed specifically for paint removal, and more specifically for graffiti removal, and which can be used in general purpose paints or surface coatings.

The present invention relates generally to alkyd or oil paint formulations, sometimes referred to as enamel paints, which contain specific enzymes, which remain inactive in an applied surface coating of the paint formulation until specifically activated by a subsequently-applied applicator solution. Both the inclusion of specific enzymes and the design of the applicator are novel and form an integral part of the invention. When the applicator is applied to finished surface coatings containing lipase enzymes, the enzymes catalyze the hydrolysis of the oil binder in the paint or surface coating by cleaving the ester bonds in the 1 and 3 positions of the triglyceride molecules. This allows for easy and rapid removal of the coating without damaging or effecting in any way the underlining surface.

The present invention also relates to methods of producing the enzyme preparation for inclusion in paint and the preparation of the rapid activator removal applicator.

### BACKGROUND OF THE INVENTION

Graffiti is usually applied on public and other buildings by way of ‘spray-can’ aerosol paints. These paints are usually alkyd and/or oil paints or oil enamels which are designed to dry rapidly. Their legal use is most helpful in ‘touch-up’ and small job applications where a rapid drying gloss finish is required. In the ‘hit and run’ world of so-called graffiti art, they are the most popular paint used.

Conventional graffiti paint removal methods usually remove or damage the base surface coating upon which the graffiti is applied. This invention is specifically designed to remove the graffiti paint only. The estimated cost of graffiti removal throughout the world is variously estimated by municipal authorities to be in the hundreds of millions of dollars each year. In many countries the call for the removal from sale of oil enamel spray (aerosol) type paint cans is very strong.

In Australia to date, the movement to ban the sale of such paints has been overcome by a voluntary paint industry initiative; which also includes the insertion of the following warning label on the paint cans: ‘IMPORTANT: Deliberate misuse of this aerosol to damage private property is a crime.’

To fully appreciate the background to this invention, it is important to understand the chemistry of both paint production and enzyme catalysis.

An enzyme is a protein with catalytic activity that is restricted to a limited set of reactions defining the specificity of the enzyme. This invention places specific water-soluble enzyme preparations into oil or alkyd oil based paints or surface coatings. The invention provides for the design of a carrier system for the enzyme that allows its incorporation into oil type paints. This is described herein below.

A simple description for a Paint follows. A more complete description can be obtained from the National Paint, Varnish and Lacquer Association, 1500 R.I. Avenue, Washington D.C. 20036 United States of America, or in ‘Paints and Surface Coatings’, Theory and Practice Second Edition. Edited by R. Lambourne and T. A. Strivens. Published by Woodhead Publishing Ltd, Abington Hall, Abington, Cambridge CB1 6AH, England 1999.

Paint is a unfirmly dispersed mixture with variable viscosity and consisting of (1) a drying oil, synthetic resin, or other film forming component called the BINDER; (2) a SOLVENT or THINNER; and (3) an Organic or Inorganic PIGMENT. The BINDER AND SOLVENT are collectively called the VEHICLE. Paints are used to protect surfaces and to provide decoration.

This invention places a specifically prepared enzyme mixture into paints which contain oils and/or alkyds as a binder type. It is also helpful to understand more of the characterization of the oils and alkyds used in paint preparation. Those skilled in the art will have a fully comprehensive understanding of oils and alkyds used in paint manufacture.

Vegetable oils and vegetable oil fatty acids continue to play an important role in surface coatings due to their availability as a renewable resource.

Oils are mixed glycerol esters of long chain monocarboxylic acids called fatty acids. The Carbon length is generally C₁₈Oils used include, Linseed Oil, Soya Bean Oil, Coconut Oil and ‘Tall Oil’. Oils are classified as Drying, Semi Drying, or Non-Drying. Table 1.0 illustrates typical composition of major oils used in surface coatings.

TABLE 1.0

| | | | | 9.12, 15 | |
|---|---|---|---|---|---|
| | Saturated | | 9.12 | Linolenic | Conjugated |
| | Acids | Oleic Acid | Linoleic Acid | Acid | Acid |
| TUNG | 6 | 7 | 4 | 3 | 90 |
| LINSEED | 10 | 20-24 | 14-19 | 48-54 | 0 |
| SOYA BEAN | 15 | 22-28 | 52-55 | 5-9 | 0 |
| CASTOR OIL | 2-4 | 90-92 | 3-6 | 0 | 0 |
| DEHYDRATED | 2-4 | 6-8 | 48-50 | 0 | 0 |
| CASTOR OIL | | | | | |
| TALL | 3 | 30-35 | 35-40 | 2-5 | 10-15 |
| COCONUT | 89-94 | 6-8 | 0-2 | 0 | 0 |

Linseed oil is a good drying oil containing more than 60% of the polyunsaturated linoleic and linolenic acids.

Alkyd resins are produced by condensation and polymerization of dihydric or polyhydric alcohols and a polybasic acid such as phthalic anhydride and usually with a drying oil modifier. These resins are described as long oil alkyds (60% oil) medium oil alkyds (40% oil) or short oil alkyds (less than 40% oil). In practical terms, the reaction of glycerol and oil/s in the production of monoglycerides in the presence of a catalyst, does not go to completion and the equilibrium distribution of species include oil, polyol and mono and diglycerides. This is important in this invention as the remaining presence of free oil/s allows, when correctly activated, the enzymatic hydrolysis of the oil to effect a gross weakening of the binder allowing for easy removal of the paint.

### OBJECTS OF THE INVENTION

It is an object of this invention to provide a paint formulation which includes an enzyme preparation which facilitates removal of surface coatings based on such paint formulations.

It is a further object of this invention to provide an alkyd or oil paint preparation which includes a lipase enzyme preparation which can be activated if and when required to facilitate removal of a surface coating or other paint deposit comprising or based on the paint preparation.

It is another object of this invention to provide a paint formulation which includes an enzyme preparation and an enzyme activator, suitable for removal of unwanted graffiti or for general paint removal when activated.

It is yet a further object of this invention to provide a surface coating composition for protecting surface structures which either have no primary surface coating, or have an existing primary surface coating which requires protection by means of the application of an outer clear surface coating.

It is yet another object of this invention to provide an enzyme preparation for inclusion in a paint formulation which facilitates removal of graffiti, or general paint removal.

It is yet again another object of this invention to provide a method of producing an enzyme preparation and an enzyme activator for inclusion in a paint formulation, and of producing a paint formulation incorporating same.

It is yet again a further object again of providing a method of graffiti removal and/or of general paint removal of a surface coating or other surface deposit of a paint formulation based on the activation of an enzyme present in the surface coating or deposit.

It is yet again another object of this invention to provide an anti-graffiti formulation which goes at least some way towards overcoming or at least minimising the prior art problems or limitations outlined above, or for providing a clear alternative choice for consumers.

These and other objects of this invention will become more apparent from the following description.

### DISCLOSURE OF THE INVENTION

According to one aspect of this invention there is provided an enzyme preparation for inclusion in a paint formulation, particularly an alkyd or oil paint formulation, together with a removal activator, which when activated is suitable for general paint removal or for specific graffiti removal.

According to another aspect of this invention there is provided a paint formulation, and especially an alkyd or oil paint formulation which includes an enzyme preparation and means for activating said enzyme, to facilitate removal of graffiti, or general paint removal, in respect of surface coatings or deposits comprising or based on the said paint formulation.

According to a further aspect of the invention, there is provided a clear paint formulation, and especially a clear alkyd or oil paint formulation, adapted to provide a clear sealer coating for both unpainted and previously painted surfaces.

### BEST MODE OF CARRYING OUT THE INVENTION

More specifically, the present invention provides a paint formulation comprising a surface coating base with one or more alkyd-, oil-, cellulose- or acrylic-type binders, and an enzyme specific to said one or more binders and adapted to be activated or catalysed when required to facilitate subsequent removal of a deposit or surface coating layer of said paint formulation.

According to a further aspect of the invention, there is provided a paint formulation of the above type, adapted to provide a clear sealer coating for both unpainted and previously painted surfaces, comprising a clear surface coating base together with one or more alkyd-, oil-, cellulose- or acrylic-type binders, and an enzyme specific to said one or more binders.

According to another aspect of the invention, there is provided a paint formulation adapted to facilitate removal of graffiti, or paint removal in general, in respect of surface coatings or deposits comprising or based on the above paint formulations.

According to yet a further aspect of the invention, there is provided a method of preparation of a paint formulation containing an enzyme which facilitates subsequent removal of a surface coating of the composition, said method comprising mixing the enzyme with a non-ionic surfactant pre-heated up to about 65° C., adding a hydrocarbon solution followed by rapid cooling of the mixture, and thereafter adding the cooled mixture to a hydrophobic paint composition and final mixing to provide an homogenous mixture of the paint formulation.

The invention further provides a method of removal of a deposit or of a surface coating of a paint formulation of the type herein described, said method comprising applying to said deposit or surface coating a further coating containing a specific activator to promote enzymatic catalysis and subsequent hydrolysis of the 1, 3 ester bonds of the triglyceride molecules present in the surface coating, thereby causing the surface coating to break down or disintegrate.

Preferably, the enzyme preparation for inclusion in alkyd and oil paints is a Lipase enzyme, such as that classified by the Chemical Abstracts Service Registry as: Lipase, triocylglycose, CAS NO 9001-62-1. Another suitable enzyme preparation for inclusion in alkyd and oil paints according to the invention is classified by the International Union of Biochemistry as EC 3.1.1.3. An example of such an enzyme according to the invention is the lipase enzyme derived from Thermomyces lanuginosus produced by submerged fermentation of a genetically modified Aspergillus oryzae microorganism, ideally with an activity of 100 KJU/g. Such an enzyme in aqueous solution with a lipolytic activity of 100 KLU/g is determined relative to an analytical standard under the following conditions: Substrate, Tributyrin. Temperature 30 degrees centigrade and pH 7.0.

Enzyme types, other than lipase, with specific catalytic activity are also useful, and are further described in the examples of this invention.

A preferred enzyme preparation according to the invention is where the aqueous enzyme is mixed with a prescribed volume of solubilized polyoxyethylene (20) oleyl ether non-ionic surfactant material. Preferably the premixed aqueous enzyme and polyoxyethylene (20) oleyl ether are further mixed with a hydrocarbon liquid. The hydrocarbon liquid may be aromatic, aliphatic or mixtures of both aromatic and aliphatic hydrocarbon liquids.

According to another embodiment, the preparation is mixed with alkyd, oil, enamel or any paint preparation containing some portion of mixed glycerol esters of long chain monocarboxylic acids (tryglycerides).

Preferably, the enzyme preparation is mixed with a paint containing an alkyd resin and such alkyd resin having been produced by condensation and polymerisation of dihydric or polyhydric alcohols and a polybasic acid such as phthalic anhydride together with a drying oil modifier.

New or existing surface structures, which have been finished with paint or other kinds of surface coatings, can also be protected by this invention by way of application of a tough clear sealer coating containing an enzyme preparation and means for activating the enzyme. A clear solution with alkyd, oil, cellulose, or acrylic type binders is applied by either spray or brush or roller on such surfaces as painted brick or block, rendered cement surfaces, tiled surfaces, stainless steel surfaces, timber and general high traffic area surfaces which can be at risk of graffiti damage.

In the event that graffiti paint is applied to such a protected surface the modified activator is capable of both removing the offending graffiti and activating the enzymes present in the clear sealer coating; thus enabling a clean removal of the graffiti and sealer coating without damage to the primary surface coating. Upon removal, the area is easily re-protected, by a fresh application of the enzyme-containing clear protective sealer coating.

The invention also provides a method of producing an activator applicator solution, wherein such solution, when applied to a painted surface containing any of the enzyme preparations described herein, will provide a chemical environment suitable for the enzymatic catalysis and subsequent hydrolysis of the 1,3 ester bonds of the tryglyceride molecules present in the paint, thereby causing the paint film to disintegrate.

Preferably, the applicator solution contains a mixture of demineralized water, sodium hypochlorite at between 50 and 60 grams per litre and sodium hydroxide at 10 grams per litre and selected alkaline salts at between 0.1 and 0.5 grams per litre and 0.3% xanthan gum derived from Xanthomonas campestris.

The invention also provides a method of removing the paint, treated as above, by vacuum equipment apparatus which produces in-situ warm to hot water and with either a brush or brushless action vacuums the paint debris into a contained vessel.

According to one preferred embodiment the enzyme preparation is a lipase enzyme derived from Rhizomucor miehei produced by submerged fermentation. The lipase enzyme can be derived from a variety of microorganisms.

The enzyme preparation according to the invention can be of a non-lipolytic type and may be of any or a mixture of the following types, protease, amylase, cellulase, or hemicellulase.

The applicant has developed various methods of producing Lipase enzyme and other enzyme solutions, which can be incorporated into any gloss solvent paint preparation. The Lipase is classified by the Chemical Abstract Services Registry as ‘Lipase,triocylglycose,CAS No. 9001-62-1’. The corresponding Enzyme Classification Number (International Union Of Biochemistry) is EC 3.1.1.3.

Other enzymes and their reactions are more fully described in the examples below.

The enzyme solution also contains but is not limited to, demineralized water, polyoxyethylene (20) oleyl ether, and liquid hydrocarbon solution/s. The activity of the enzyme preparation is expressed in Kilo Lipase Units (KLU). The invention also includes the materials and method of manufacture of an applicator suitable for activating the unactivated enzyme solution described above and contained within a dried surface coating. The enzyme preparation is designed to operate at a pH of around 11.0. Typical paint preparations described previously have a pH of around 6.8. The enzyme preparation can be activated at a temperature of between 10° C. and 40 C.

The activator solution contains warm to hot water—which can be prepared in the special device described below, sodium hydroxide, sodium hypochlorite, alkaline salts, xanthan gum, methylene chloride and sequestering agents and surfactants.

Upon activation the enzyme preparation hydrolyses the ester bonds in the 1,3 position of the tryglyceride molecules present in the paint binder and weakens the paint film so that cleaning and removal can be affected by the apparatus described below. Preferably, the invention also relies upon utilizing a vacuum type water cleaner, similar to those used in carpet cleaning, for one-step removal of the paint film weakened as described above. This apparatus produces in-situ warm to hot water and with either a brush or brushes action vacuums the paint debris into a contained vessel and minimizes clean up time.

### DETAILED DESCRIPTION OF THE INVENTION

The nature of the present invention may be more clearly understood from the following preferred but non-limiting examples.

### EXAMPLE 1

A black enamel paint containing linseed oil in the binder component of the vehicle was selected.

In a preferred embodiment of this invention an enzyme solution was prepared for inclusion into this black enamel paint as now described.

A lipase enzyme solution, containing lipase enzymes derived form Thermomyces lanuginosus produced by submerged fermentation of a genetically modified Aspergillus oryzae microorganism, with an activity of 100 KJL/g was selected. This enzyme preparation is a 1,3- specific lipase, in that it cleaves the ester bonds 1 and 3 of a triglyceride. This enzyme protein has an IUB Number of 3.1.1.3 and a CAS Number of 9001-62-1 and an EINECS Number of 232-619-9. This enzyme has maximum activity at pH 11.0. The pH of the selected black enamel paint is 6.8. This enzyme has optimum activity at 38° C. The enzymes as so far described are contained in an aqueous solution containing between 1-10% w/w protein enzymes. The following description describes how to incorporate this aqueous solution into a hydrophobic enamel paint.

A quantity sufficient of polyoxyethylene (20) oleyl ether is heated to, between 28° C. and 65° C. To this heated non-ionic surfactant is added a volume of aqueous lipase enzyme preparation as previously described. This is well mixed. To this mixture is added a 100% hydrocarbon solution with a boiling point in the range 155° C.-165° C. and a relative density of 0.85-0.91. This is well mixed and rapidly cools the mixture. This complete solution is now capable of being added to the hydrophobic enamel paint. A prescribed aliquot of this solution is added to 1000 mls of the black enamel paint and well mixed in.

This paint-enzyme preparation is then applied by fine horse hair brush to wooden, steel and concrete surfaces and allowed to dry in the normal way. Untreated black enamel paint is applied to adjoining portions of the wood, steel, concrete surfaces and allowed to dry.

The rapid enzyme activator solution is prepared in the following way.

To demineralized water is added Sodium Hypochlorite at between 50 and 60 grams per litre and Sodium Hydroxide at 10 grams per litre and selected Alkaline salts between 0.1 and 0.5 grams per litre. To this combined and well mixed solution is added up to 0.3% xanthan gum derived from Xanthomonas campestris. Sufficient non-ionic surfactant is added to improve surfactant qualities of the mixture. The final pH of the mixture is adjusted to pH 11.0.

The applicator is applied at ambient temperatures to the prepared painted surfaces described above. The applicator is carefully applied to the total painted areas. Due to its high viscosity the applicator can remain on vertical surfaces for considerable periods. The combination of pH, penetration, time and temperature work to activate the dormant lipase enzyme catalysts. At various times frames, commencing at 20 minutes, a carpet cleaner type vacuum cleaner capable of heating water and scrubbing and vacuuming surfaces is employed to wash and scrub all treated and non treated painted surfaces. The water temperature is 39° C.

The enzyme contained painted surfaces are easily removed. The non-enzyme containing painted surfaces remain intact and retain gloss and colour.

### EXAMPLE 2

An enzyme protein described as Lipase, in an aqueous solution and with an IUB number of 3.1.1.3 and a CAS number of 9001-62-1 and an EINECS number 232-619-9 and a prescribed lipolytic activity of 100 KLU/g was selected. This activity is determined relative to an analytical standard under the following conditions:

| Substrate: | Tributyrin |
|---|---|
| Temperature: | 30° C. |
| pH: | 7.0 |

Tribuyrin is described in the Merck Index, Twelfth Edition, and Published by Merck Research Laboratories, Division of Merck and Co. Inc., Whitehouse Station, N.J., USA 1996, as follows:
Tributyrin. Butanoic acid 1,2,3-propanetriyl ester; glyceryl Tributyrate. C₁₅H2₆O₆ Mol. Weight 302.37. C, 59.58% H, 8.67% O, 31.75% (C₃H₇COO)₃C₃H₅. Prepared by esterification of glycerol with excess butyric acid. Oil liquid. Bitter taste. Insoluble in water.

The enzyme described above is a 1,3- specific lipase that cleaves the ester bonds in positions 1 and 3 of a triglyceride. The enzyme has a lipolytic activity of between 60 and 100% in a temperature range of between 10° C. and 60° C. Optimum activity is between 30° C. and 40° C. Optimum lipolytic activity is found at pH 11.0.

The enzyme described is classified as non-toxic. The enzyme solution is biodegradable.

By exhaustive experimentation, the inventors discovered that relatively minor amounts of the described enzyme can be contained in the paint solution to be effective. Effectiveness is the ability that when an activator is applied the contained enzymes weaken the binding capability within a paint film so that easy removal is facilitated.

In this example an amount equivalent to 0.16% of active enzyme in a litre of paint solution is selected for inclusion in a non-ionic hydrocarbon carrier. So that no greater than 1% of total additive is added to a finely balanced paint formulation sufficient polyoxyethylene (20) oleyl ether is heat sobulised to absorb the enzyme preparation and mixed well.

To the cooled and well mixed mixture is added sufficient liquid hydrocarbon solution with a boiling point of between 155° C.-165° C., and a relative density of 0.85-091. The solution is added to black enamel paint so that the total addition on a weight/weight basis is less than 1%.

### EXAMPLE 3

It is most important that the enzyme preparation is delivered to the paint emulsion in as colourless condition as possible. Paint formulators are most careful in pigment quality and quantity selection. In coloured paints the slightest variation can cause significant colour and tone changes.

The inventors have discovered a means of first incorporating the enzyme stock solution in a non-ionic surfactant and then combining this solution with a hydrocarbon solvent which enables the total liquid to be successfully incorporated into an oil type paint. The solution is colourless to very slight white-yellow.

The rate of addition of enzyme effectively delivered by this solution, created no colour change in several samples of paint, when measured by spectrophotometer.

A selected polyoxyethylene (20) oleyl ether is employed as the non-ionic surfactant. This material is semi solid at temperatures of up to 25° C. The first difficulty to overcome in this process is to liquefy the surfactant at temperatures which will not activate the lipase enzyme. It was discovered that by heating the surfactant and enzyme solution to 25° C., mixing vigorously and adding rapidly the required volume of hydrocarbon liquid, the enzyme mixture could be prepared without loss of activity. The mixing must be vigorous. The hydrocarbon liquid rapidly cools the mixture.

The enzyme solution can be either added to a finished paint or incorporated in the build of the paint. In this example, as in the previously cited examples, the enzyme solution is added to a finished black enamel paint. Addition is at ambient temperature and with moderate, non-foaming agitation.

The enzyme-included paint is applied to various surface types and tested by application of the activator, described previously.

### EXAMPLE 4

In paint manufacture care is given to the properties known as sedimentation and flocculation. The pigments employed usually have a density greater than the resin solution of the paint. Therefore under the influence of gravity pigments will tend to settle according to Stokes law. This factor is considered carefully in the selection of the hydrocarbon material used to disperse the pigment. Aliphatic or aromatic hydrocarbons may be used.

Either Aliphatic hydrocarbon solvent (white spirit) or Aromatic hydrocarbon solvent (toluene) may be used in this invention for incorporation of the enzyme into paint. Toluene (methylbenzene) is slightly soluble in water. This is helpful in the context of the invention.

The paint manufacturer may prefer white spirit to toluene, for greater pigment absorption and the development of a stronger dispersion.

As in previous examples, the selected Lipase enzymes are incorporated into a non-ionic surfactant and either aromatic or aliphatic hydrocarbons, or mixtures of both aromatic and aliphatic are used for dispersion into the built paint. The use of surfactant further ensures that this invention does not alter the pre-existing sedimentation characteristics present in the paint.

The addition of the co-mixture does not alter the rheological characteristics of the paint.

### EXAMPLE 5

In biological terms the usual tendency for natural decay mechanisms is for a lowering of pH values to acidic. This phenomenon is an important consideration in this invention. A paint or surface coating containing the enzyme systems, so far described in the above examples, must retain its usual characteristics when used for legitimate purposes. The enzyme systems employed are deliberately selected to operate in high alkaline, high pH conditions. Unless the paint system is subjected to constant caustic bombardment the enzyme mechanism will not be activated. If such an eventuality were to occur naturally it is unlikely that any paint system could maintain gloss, appearance and durability for prolonged periods.

To further safeguard legitimate paint applications the enzyme system can only be activated to catalysis conditions by complex alkaline systems and slightly elevated temperatures.

### EXAMPLE 6

New or existing surface structures, which have been finished with paint or other kinds of surface coatings, can be protected by this invention by way of application of a tough clear sealer coating containing a specially prepared enzyme solution according to the invention. A clear solution with alkyd, oil, cellulose, or acrylic type binders is applied by either spray or brush or roller on such surfaces as painted brick or block, rendered cement surfaces, tiled surfaces, stainless steel surfaces, timber and general high traffic area surfaces which can be at risk of graffiti damage.

Cellulose nitrate (nitrocellulose) containing lacquers and acrylic lacquers, as well as vinyl preparations containing either organic esters of cellulose or cellulose derivatives such as hydroxy ethyl cellulose, can be prepared which contain enzyme mixtures, which when activated are capable of weakening these types of binders. Once weakened removal of the sealer paint film proceeds as previously described.

Cellulase or hemicellulase enzymes or combinations of each can be utilised in the enzyme preparation included in the clear sealer coating/s.

Cellulase enzymes derived from either Aspergillus niger or Trichoderma viride capable of decomposing cellulosic polysaccharides into smaller fragments are selected for inclusion in an admixture, which also contains lipase enzymes, non-ionic surfactant and hydrocarbon solutions.

A novel cellulase enzyme derived from a thermophilic soil fungus, Thielatia terrestris, is selected for inclusion in a clear surface coating, this enzyme is only activated to catalysis by high temperature. This feature is particularly suitable for graffiti removal as described previously hereinabove.

An activator containing sodium hydroxide, sodium hypochlorite, alkaline salts, methylene chloride, n-methyl pyrrolidone, xanthan gum and non-ionic surfactant, is added to very hot water and mixed thoroughly. The temperature of the water is greater than 78° C. Apparatus previously described, applies the activator to graffiti paint damaged surfaces. Such surfaces having been previously coated with the clear sealer coating already described. The activator will remove the graffiti paint, irrespective of its type or origin. The enzyme contained clear sealer coating beneath the graffiti damage, will be weakened by the action of the enzyme catalysis triggered by the activator. A complete removal of the graffiti and the enzyme containing clear sealer coating can be effected without damage to the primary surface coating. Once dry, the primary surface can again be protected by application of the clear enzyme/s containing sealer protective coating.

Although the invention has been described with reference to preferred embodiments and examples, the present invention has been shown and it will be apparent to those having ordinary skill in the art that a number of changes, modifications or alterations to the invention described herein may be made, none of which depart from the spirit of the present invention. All such changes, modifications, and alterations should therefore be seen as being within the scope of the present invention.

It should be appreciated that the present invention provides a substantial advance in paint formulations and the removal thereof providing all of the herein-described advantages without incurring any relative disadvantages.

The words “comprise”, “comprises” and “comprising”, as used herein, are used in the inclusive sense of “having” or “including”, and not in the exclusive sense of “consisting only of”.

1-19. (canceled)

20. A paint formulation comprising:
(a) a mixture of a surface coating base containing at least one binder, wherein the binder is a member selected from the group consisting of alkyd resins, oils, cellulose, acrylic resins, and mixtures of same; and
(b) at least one enzyme specific to the at least one binder that can be activated to facilitate subsequent removal of a coating of the paint formulation from a surface on which the coating has been deposited.

21. The paint formulation of claim 20, which additionally contains a pigment.

22. A clear sealer coating for unpainted or previously painted surfaces comprising a formulation according to claim 20, wherein the formulation comprises:
(a) a mixture of a surface coating containing at least one binder, wherein the binder is a member selected from the group consisting of alkyd resins, oils, acrylic resins and mixtures of same;
(b) at least one enzyme specific to said at least one binder which can be activated to facilitate subsequent removal of the clear sealer coating from a surface on which the coating has been deposited.

23. The paint formulation of claim 20, wherein the enzyme is a lipase enzyme.

24. The paint formulation of claim 23, wherein the enzyme is a Lipase enzyme, classified by the Chemical Abstracts Service Registry as: Lipase, tricylglycose, CAS NO 9001-620-1.

25. The paint formulation of claim 23, wherein the enzyme is one classified by the International Union of Pure and Applied Chemistry as EC 3.1.1.3.

26. The paint formulation of claim 23, wherein the enzyme is derived from Thermomyces lanuginosus produced by submerged fermentation of a genetically modified Aspergillus oryzae microorganism with an activity of about 100 KLU/g.

27. The paint formulation of claim 23, wherein the enzyme is derived from Rhizomucor miehei produced from submerged fermentation.

28. The paint formulation of claim 26, wherein the lipolytic activity of about 100 KLU/g is determined relative to an analytical standard under a set of conditions wherein the reaction substrate is tributyrin, and the reaction occurs at 30° C., and at pH 7.0.

29. The paint formulation of claim 20, wherein said at least one enzyme is mixed with solubilized polyoxyethylene (20) oleyl ether non-ionic surfactant.

30. The paint formulation of claim 28, wherein said mixture of said at least one enzyme with a surfactant is mixed with a liquid hydrocarbon.

31. The paint formulation of claim 30, wherein the liquid hydrocarbon is a member selected from the group consisting of aromatic hydrocarbons, aliphatic hydrocarbons, and mixtures thereof.

32. The paint formulation of claim 20, wherein the enzyme is mixed with a paint preparation of a type selected from the group consisting of alkyd, oil, enamel, and paint preparations comprising mixed glycerol esters of long-chain monocarboxylic acids (triglycerides).

33. The paint formulation of claim 20, wherein the enzyme mixture is mixed with a coating base comprising an alkyd resin, wherein the resin has been produced by condensation and polymerization of a polyhydric alcohol and a polybasic acid, and with a drying oil modifier.

34. The paint formulation of claim 20, wherein the enzyme is of a member selected from the group consisting of a protease, amylase, cellulase and hemicellulase enzymes.

35. The clear paint formulation of claim 22, wherein the enzyme is a member selected from the group consisting of cellulase, hemicellulase, enzymes and combinations thereof.

36. An activator solution for application to a surface painted with a formulation according to claim 20, comprising a mixture of sodium hypochlorite, sodium hydroxide, alkaline salts and a xanthan gum derived from Xanthomonas campestris which when applied to said painted surface will cause said paint to degrade to a point permitting easy removal.

37. A kit comprising a formulation according to claim 20, and an activator comprising a mixture of sodium hydroxide, sodium hypochlorite, alkaline salts and a xanthan gum derived from Xanthomonas campestris with said activator being separately maintained until removal of the paint formulation from said paint coated surface is desired.

38. A method for removal of at least a portion of a surface coating comprising the paint formulation of claim 20, comprising the step of applying a further coating comprising an activator formulation containing an enzyme specific to said at least one binder in said formulation to at least portion of the surface coating, wherein the activator formulation promotes the catalysis and subsequent hydrolysis of 1, 3 esters bonds of triglyceride molecules present in said surface coating, thereby causing the degradation thereof.

39. The method of claim 38, wherein the activator composition comprises demineralized water; sodium hypochlorite, present in said composition in an amount of between 50 to 60 grams per liter; sodium hydroxide, present in said composition in an amount of about 10 grams per liter; and 0.35 xanthan gum, wherein the xanthan gum is derived from Xanthomonas campestris.

40. The method of claim 38, comprising the additional step of treating the degraded portion of the surface coating with vacuum equipment apparatus that produces hot water and is effective to remove the degraded portion of the surface coating into a contained vessel.

41. A method of preparation of a paint formulation comprising an enzyme that facilitates subsequent removal of a surface coating of the paint formulation from a surface onto which the formulation has been deposited, comprising the steps of mixing the enzyme with a non-ionic surfactant pre-heated to about 65° C.; adding a hydrocarbon solution to the mixture and leading to rapid cooling thereof; adding the cooled mixture to a hydrophobic paint composition; and subjecting the formulation to final mixing to provide a homogeneous mixture of the paint formulation
